(19) United States
(12) Patent Application Publication
(10) Pub. No.: US 2020/0054288 A1
(43) Pub. Date: Feb. 20, 2020

(54) Method, Device and System for Detection, Prediction and Classification of Physical Activities Using IMU Sensors Placed on Wearable Fabrics for Knees

(71) Applicant: Volkan Vural, San Diego, CA (US)

(72) Inventor: Volkan Vural, San Diego, CA (US)

(21) Appl. No.: 16/543,518

(22) Filed: Aug. 17, 2019

Related U.S. Application Data
(60) Provisional application No. 62/719,650, filed on Aug. 19, 2018.

(51) Int. Cl. A61B 5/00; G01P 15/18; G06N 3/08; A61B 5/11

(52) U.S. Cl. CPC A61B 5/7267; G01P 15/18; G06N 3/08; G06N 3/0445; A61B 5/1123; A61B 5/1121; A61B 5/6828

(57) ABSTRACT

Characterization of physical activity of a person by analyzing sensor measurements acquired by IMUS placed on wearable fabrics such as for knees. The system provides detection and/or prediction of physical activities. Activity classification is achieved by pattern analysis methods running on appropriate computing platforms, including, but not limited to mobile phones, mobile devices, tablets, laptops, PCs, servers, fitness tracking devices, or microcontrollers located on a preferred embodiment, and/or like. Classified activities can be used for reporting daily exercises as well as to estimate calorie expenditure, and to serve for personalized fitness monitoring and coaching purposes.

### CROSS REFERENCE TO RELATED APPLICATIONS

This application claims the benefit of U.S. Provisional Patent Application No. 62/719,650, filed on Aug. 19, 2018 and entitled “METHOD, DEVICE AND SYSTEM FOR CLASSIFICATION OF PHYSICAL ACTIVITIES USING IMU SENSORS PLACED ON WEARABLE FABRICS FOR KNEES” pursuant to 35 USC 119, which application is incorporated fully herein by reference.

### REFERENCES

### U.S. Patent Documents

U.S. Pat. No. 8,909,543

U.S. Pat. No. 9,352,207

U.S. Pat. No. 9,081,889

U.S. Pat. No. 10,109,175

U.S. Pat. No. 8,894,548

U.S. Pat. No. 8,849,610

U.S. Pat. No. 9,011,292

U.S. Pat. No. 8,543,185

U.S. Pat. No. 9,148,483

U.S. Pat. No. 8,951,165

### TECHNICAL FIELD AND BACKGROUND

### Field of the Invention

The disclosure generally relates to physical activity detection, prediction and, in particular, to the use of machine learning techniques to analyze signals acquired using IMU sensors on wearable knee fabrics.

### Background

Development of wearable devices for physical activity detection is known for a number of applications. For example, U.S. Pat. No. 8,909,543, filed on Dec. 9, 2014 describes a movement detection method to encourage physical activity. A similar purpose is addressed by U.S. Pat. No. 9,352,207, filed on May 31, 2016, where a shoe-mounted and/or a wrist-worn accelerometer device is utilized to classify several physical activities by analyzing sensor outputs using frequency-domain signal processing techniques.

In the prior art, a generic framework for activity management is described in U.S. Pat. No. 9,081,889, filed on Jul. 14, 2015. Similarly, a notification system triggered by the detection of physical activity is described in U.S. Pat. No. 10,109,175, filed on Oct. 23, 2018. In U.S. Pat. No. 8,894,548, filed on Nov. 25, 2014, systems and methods for generating performance data based on an individual's performance during a physical activity are disclosed.

Methods, devices, systems and computer programs for consolidating overlapping sensor data by multiple physical activity monitoring sensors are described in, for instance, U.S. Pat. No. 8,849,610, filed on Sep. 30, 2014, and in U.S. Pat. No. 9,148,483, filed on Sep. 29, 2015. A wearable athletic information device is described in U.S. Pat. No. 9,011,292, filed on Apr. 21, 2015. Systems and methods for physical activity monitoring using output signals from a multitude of multimodal sensors are described in U.S. Pat. No. 8,543,185, filed on Sep. 24, 2013. A physical activity monitoring device that assists in personal training is disclosed in U.S. Pat. No. 8,951,165, filed on Feb. 10, 2015. In U.S. Pat. No. 9,597,567, filed on Mar. 21, 2017, a smart sport device with a hidden Markov model-based activity recognition module is described for analyzing signal outputs from a multitude of sensors including microphone arrays and cameras.

In the prior art, methods, devices and systems are disclosed for physical activity detection depending on the analyses of output signals of a number of multimodal sensors. These require high computational power. Moreover, they do not support features like fully automated tracking and personalization of physical activities. Therefore, there is a need for physical activity detection, prediction and classification method and system that are not constrained by the foregoing limitations of the prior art.

### SUMMARY OF THE INVENTION

The present invention is a physical activity detection, prediction and classification method and system based on machine learning techniques that analyze signals acquired using IMU sensors placed on a wearable fabric, such as wearable knee fabrics. A system using the invention provides a more accurate, fully automated and personalized alternative to fitness trackers which are currently used to detect and analyze physical activities. The method of the invention processes output signals from one or more IMU sensors (“IMU data”) placed on or integrated with wearable fabrics such as for knees. One preferred embodiment of the invention uses recurrent neural networks to detect and classify physical activities.

The invention discloses a method and system for predicting and classifying physical activities in an automated fashion using the IMU data. IMU data gathered from one or more IMU sensors, which may be provided as a MEMS device capable of measuring a force, angular rate, acceleration and the orientation of a body, using a combination of accelerometers, gyroscopes, and magnetometers, is installed or integrated into wearable fabrics for, e.g., knees, and fed into physical activity classification method.

In one aspect, a type of an artificial neural network architecture, namely, long-short-term-memory (LSTM) is used to classify time-series IMU data composed of acceleration and gyroscope sensor outputs.

In another aspect, IMU data is processed to infer a user's way of practicing physical activities. Additionally, fully automated physical activity tracking is made possible by analyzing the IMU data using supervised or unsupervised learning techniques.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a preferred embodiment, referred to as “Wearable IMU Data Collecting and Transmitting Device”.

FIG. 2 is the multi-functional hardware module comprising an IMU sensor, processor, memory, transceiver and a display.

FIG. 3 depicts the physical activity analysis method, which can be implemented on a mobile device.

FIG. 4 shows physical activity classifier.

### DETAILED DESCRIPTION OF PREFERRED EMBODIMENT OF THE INVENTION

The present invention is an innovative device and system for predicting and classifying physical activities automatically by analyzing IMU data obtained from wearable sensors placed on a knee or other body-worn fabrics.

There are different types of physical activities with varying characteristics and involvement of different muscles and/or muscle groups. Practicing different activities results in generation of IMU data with varying temporal characteristics. By analyzing the IMU data, the present invention automatically detects and classifies physical activity.

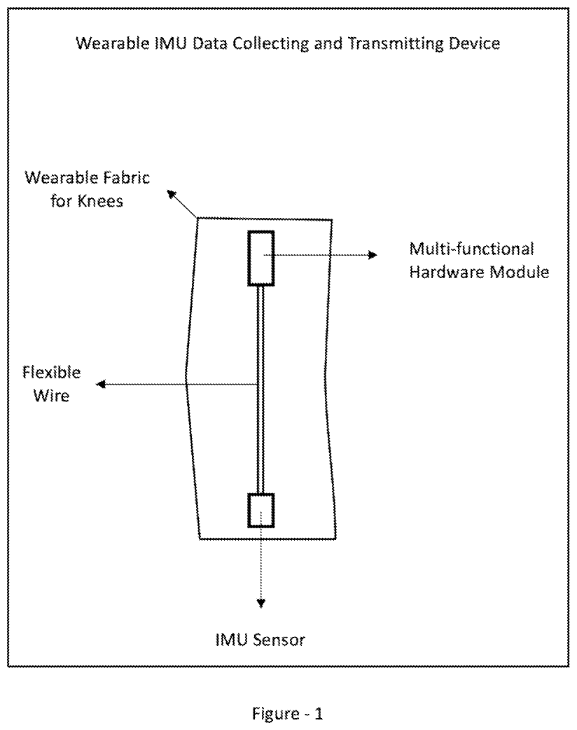

Referring now to the drawings, a preferred embodiment, referred to as “Wearable IMU Data Collecting and Transmitting Device”, is presented in FIG. 1. The device comprises a multi-functional hardware module and an IMU sensor connected with a flexible (elastic) wire mounted on a wearable fabric for knees. In a preferred embodiment, the IMU Data Collecting and Transmitting Device may be attached/woven to a knee sleeve. When worn by the user, the multi-functional hardware module and the IMU sensor preferably stays on upper and lower parts of the knee.

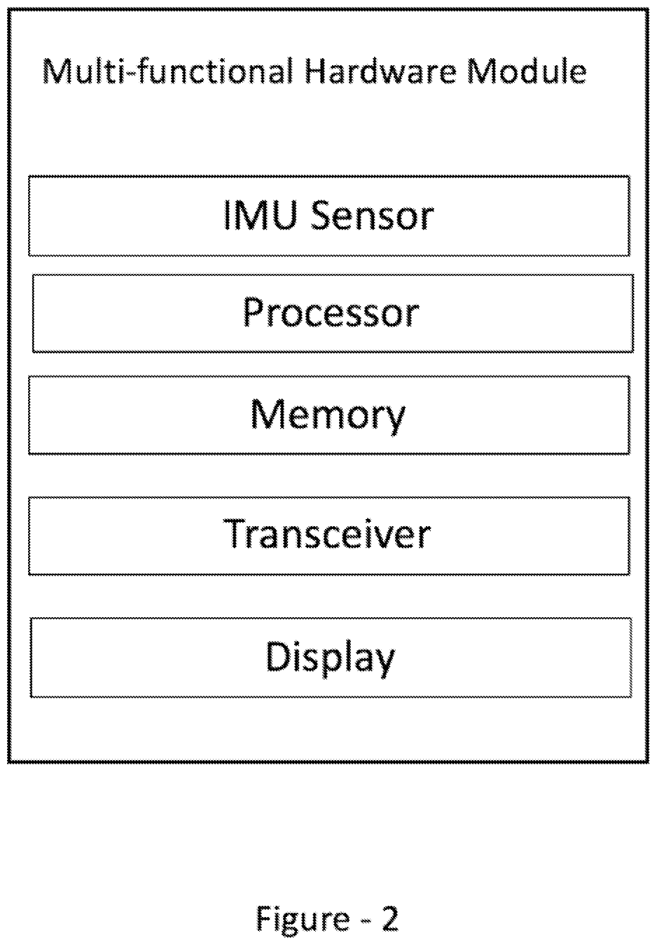

Turning now to FIG. 2, the inner structure of the multi-functional hardware module is presented. It is comprised of an IMU Sensor feeding acceleration and gyroscope data to a processor configured to process the IMU data to identify and output one or more user-defined characteristics, classifications and/or attributes in the data. By analyzing the IMU data, the processor outputs the classification results which may then be recorded in a system memory, or sent outside via a transceiver and/or displayed.

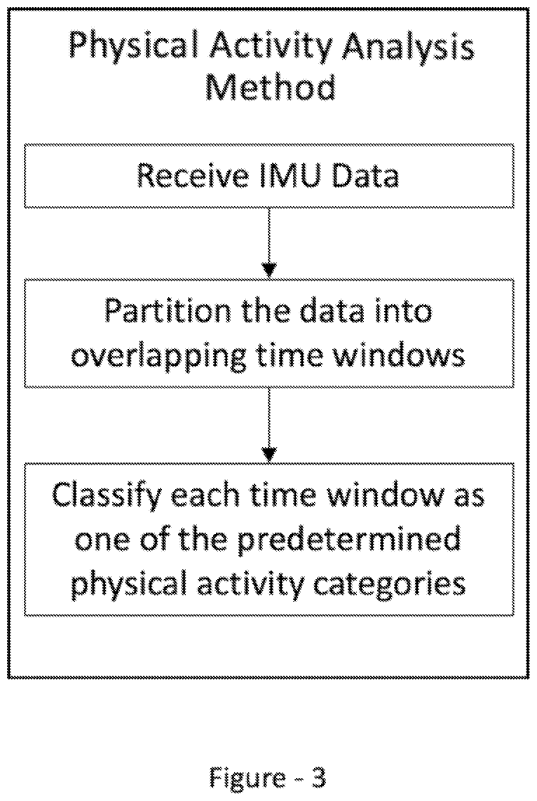

The physical activity classification method, which can be implemented and run on a processor and/or on a mobile device, is presented in FIG. 3. In a preferred embodiment, IMU sensor output signal may be sampled at 10 Hz. Received IMU data is preferably partitioned into half-overlapping time windows of a length of two-seconds. Data within each time window is classified as one of the predetermined physical activity categories selected by the user.

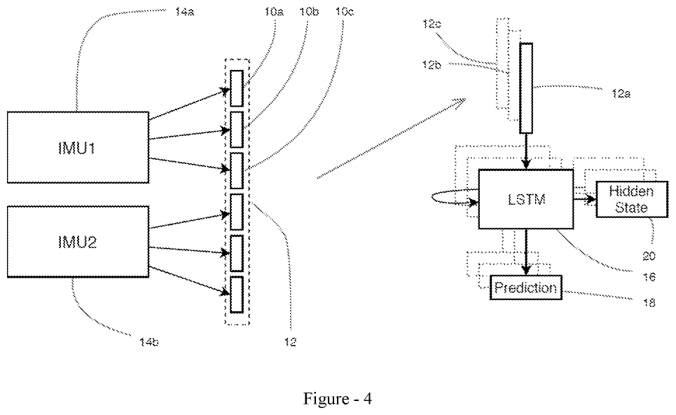

Turning now to FIG. 4, the classification algorithm is explained. Each exemplar IMU device (14a, 14b) comprises an acceleration sensor and gyroscope sensor with three dimensions for x, y and z axes (10a, 10b, 10c). Concatenating these vectors, the system obtains a 12-dimensional array (12) to represent all the sensory observations in a single time step.

Activity prediction is achieved by supervised learning using an LSTM architecture (16). Labels are assigned for every timestep and prediction is realized using a categorical distribution serving as the statistical model (18). During a training phase, the LSTM model is fed with hidden states of the previous iteration (20), and 20 consecutive observation vectors, each of which is sampled at exemplar 10Hz. While in evaluation/training, the model is fed sequentially (12a, 12b, 12c). In FIG. 4, 10a 10b 10c show X axes, Y axes and Z axes of accelerometer and gyroscope sensor at a single time step, respectively.

Element 12 is the concatenated vector of sensor readings. 12a, 12b and 12c depicting sequential observation vectors corresponding to the sample vectors at the current, previous and one before previous time instants. Elements 14a and 14b show IMU sensors equipped with an accelerometer and gyroscope. Element 16 corresponds to the recurrent artificial neural network (LSTM module). Element 18 is the softmax output (prediction) of the neural network, and element 20 is the hidden state of the LSTM module.

While the invention has been described in terms of preferred embodiments, those skilled in the art will recognize that the invention can be practiced with modification within the scope of the appended claims.

I claim:

1. A knee-worn sensor system for the purpose of detection, prediction and classification of physical activities of an individual:
comprising an IMU sensor, a flexible electrically conductive cable and a multi-functional hardware module.

2. The sensor system of claim 1 wherein the multi-functional hardware module comprises an IMU sensor and a processor wherein the processor is configured to analyze one or more physical activities of the individual.

3. The sensor system of claim 1 or claim 2 wherein an accelerometer and a gyroscope output of IMU sensors are received, sampled and partitioned into overlapping fixed-sized sample windows to form an IMU data set.

4. The sensor system of claim 3 wherein IMU data corresponding to each time window is classified into a type of physical activity performed by the individual.

5. The sensor system of claim 3 or claim 4 wherein the classification step comprises a sequential analysis of IMU data within each time window using a recurrent supervised learning algorithm.

6. The sensor system of claim 1 wherein the multi-functional hardware module comprises a memory, a transceiver and a display and is configured to store, transmit and/or display IMU data and/or activity type.
